(19) United States
(12) Patent Application Publication
(10) Pub. No.: US 2020/0187896 A1
(43) Pub. Date: Jun. 18, 2020

(54) APPARATUS AND METHOD FOR ASSESSING UTERINE PARAMETERS

(71) Applicant: Samsung Electronics Co., Ltd., Suwon-si, Gyeonggi-do (KR)

(72) Inventors: Nitin SINGHAL, Bangalore (IN); Srinivas Rao KUDAVELLY, Bangalore (IN)

(21) Appl. No.: 16/618,634

(22) PCT Filed: Jun. 1, 2018
(86) PCT No.: PCT/KR2018/006303

§ 371 Date: Dec. 2, 2019

(30) Foreign Application Priority Data
Jun. 2, 2017 (IN) 201741019461
Nov. 29, 2017 (IN) 2017 41019461

(51) Int. Cl. A61B 8/08; A61B 8/06; A61B 8/12

(52) U.S. Cl. CPC A61B 8/0866; A61B 8/0858; A61B 17/435; A61B 8/06; A61B 8/12; A61B 8/5223

(57) ABSTRACT

A method of assessing uterine parameters via a apparatus including a processor includes determining, by the processor, at least one uterine parameter based on one or more ultrasound images of a uterus of a subject, the one or more ultrasound images being obtained on predefined days within an IVF cycle of the subject, tracking, by the processor, a change in the at least one uterine parameter, and predicting, by the processor, success of embryo implantation based on the change in the at least one uterine parameter.

### TECHNICAL FIELD

Embodiments herein relate to obstetrics and gynecology, and more particularly, to methods and systems for providing clinical aid to predict the success of embryo implantation during In-Vitro fertilization (IVF).

### BACKGROUND ART

Embryo implantation is one of the critical steps in the reproductive process. Embryo implantation is a biological phenomenon in which the blastocyst becomes intimately connected to the endometrial surface to form the placenta. Successful embryo implantation depends on multiple factors, including embryo quality, uterine (endometrial) receptivity, implantation location and time, and so on. The uterus can be considered as receptive when it is ready for embryo implantation. This occurs around the 19-21 day period in the menstrual cycle of a fertile woman. The lack of synchronization between the embryo, which is ready to be implanted, and uterine receptivity is one of the causes of failure of embryo implantation. Inadequate uterine receptivity can be an unsolved problem in reproductive medicine and is considered as a major cause of infertility in otherwise healthy women.

Typically, the chances of multiple pregnancies/gestations are higher in IVF. The problem of multiple gestations is that they increase maternal and fetal risks. Increased medical, societal, and regulatory attention is provided for controlling multiple gestations. Improved medical procedures such as lower hormonal dosages, single embryo transfer, guidance during embryo transfer, and so on, have all contributed to the reduction in multiple pregnancies. With advancement in cryobiology and elective single embryo transfer, it is necessary to improve pregnancy rates in IVF cycles.

### DISCLOSURE OF INVENTION

### Technical Problem

Uterine receptivity can be associated with endometrial receptivity. Endometrial receptivity plays a crucial role in the establishment of a healthy pregnancy in IVF. It can be shown that controlled ovarian hyper-stimulation has a significant impact on the uterine lining, which leads to different results for the predictive value of endometrial factors measured on different cycle days.

Existing methods of determining uterine receptivity are based on endometrial factors such as endometrium thickness, endometrium volume, endometrial pattern, and sub-endometrial blood flow. However, there is no clear consensus on whether the endometrial factors are appropriate for predicting the outcome of IVF, and which endometrial factors can be used, if appropriate, for predicting the outcome of IVF.

### Solution to Problem

The technical solution of the embodiments herein is to provide methods and systems for providing aid to clinicians for the assessment of uterine parameters.

Another technical solution of the embodiments herein is to provide aid to clinicians for the assessment of uterine parameters for IVF.

Another technical solution of the embodiments herein is to quantify the uterine parameters of a subject, such as endometrium thickness, a endometrium volume, junctional zone thickness, junctional zone volume, uterine blood flow, and other relevant uterine parameters, during the stimulation cycle of the IVF of the subject and to track the quantified uterine parameters throughout the stimulation cycle.

Another technical solution of the embodiments herein is to track the quantified uterine parameters during predefined days of the stimulation cycle of the IVF cycle of the subject and compare the quantified uterine parameters of the subject with the uterine parameters of other subjects during the stimulation cycle of the IVF of the other subjects.

Another technical solution of the embodiments herein is to determine the implantation location of the embryo, the day to implant the embryo, and the grade embryo receptivity of the uterus and to predict the success of embryo transfer, based on the quantified uterine parameters tracked throughout the stimulation cycle of the IVF.

Another technical solution of the embodiments herein is to retrieve the quantified uterine parameters of a plurality of subjects and build nomographs based on the rate of change of the quantified uterine parameters.

Another technical solution of the embodiments herein is to obtain a mid-coronal plane view of the uterus from a three-dimensional ultrasound image of the uterus to visualize the multi-layers of the uterus of the subject and thereby segment different layers of the uterus of the subject.

Another technical solution of the embodiments herein is to perform automated segmentation of different uterine layers in the three-dimensional ultrasound image of the uterus of the subject.

Another technical solution of the embodiments herein is to obtain enhanced visualization of blood perfusion inside the junctional zone.

Another technical solution of the embodiments herein is to obtain enhanced visualization of the implantation location marker.

Another technical solution of the embodiments herein is to determine a hormonal dosage based on the rate of change of quantified uterine parameters.

Another technical solution of the embodiments herein is to diagnose adenomyosis, endometriosis, and other related uterine pathologies based on the quantified uterine parameters.

Another technical solution of the embodiments herein is to classify different types of uterine shapes for detecting structural anomalies in the uterus of the subject.

### Advantageous Effects of Invention

A method and an apparatus according to the embodiments herein provide aid to clinicians for the assessment of uterine parameters during IVF.

According to the embodiments herein, the time taken to perform a scan assessment by a medical professional and operator dependency are reduced.

A method and an apparatus according to the embodiments therein, provide appropriate implantation location and implantation time for embryo transfer.

### BRIEF DESCRIPTION OF DRAWINGS

The disclosure is illustrated in the accompanying drawings, through out which like reference letters indicate corresponding parts in the various figures. The embodiments herein will be better understood from the following description with reference to the drawings, in which:

FIG. 1A depicts the anatomy of the female reproductive system;

FIG. 1B depicts different layers of the uterus;

FIG. 2 depicts the timeline of the IVF process, according to embodiments;

FIGS. 3A and 3B depict changes in a junctional zone on predefined days of the IVF cycle, according to embodiments;

FIG. 4 depicts various units of an apparatus for analyzing uterine parameters post IVF, according to embodiments;

FIG. 5 depicts a mid-coronal plane view of a uterus from a 3D trans-vaginal ultrasound, according to embodiments;

FIG. 6 depicts segmentation of a junctional zone of the uterus from the mid-coronal plane view, according to embodiments;

FIGS. 7A-7C depict results of segmentation of the uterine cavity and junctional zone, according to embodiments;

FIGS. 8A-8E depict tracking of the junctional zone during IVF cycle, according to embodiments;

FIGS. 9A and 9B depict enhanced visualization of perfusion in the junctional zone, according to embodiments;

FIG. 10 shows a flowchart of a method for assessing uterine parameters via the apparatus, according to embodiments;

FIG. 11A shows an example a flow chart of a method of needle path tracking for embryo implantation, according to embodiments;

FIG. 11B shows an example scenario depicting tracking of a needle path for embryo implantation, according to embodiments; and

FIG. 12 shows a graph depicting a nomograph obtained from tracked uterine parameters, according to embodiments.

### BEST MODE FOR CARRYING OUT THE INVENTION

Embodiments herein provide methods and systems for providing clinical aid for assessing the uterine parameters of a subject.

In accordance with an aspect of the disclosure, a method of assessing uterine parameters via an apparatus including a processor includes determining, by the processor, at least one uterine parameter based on one or more ultrasound images of the uterus of a subject obtained on predefined days within the IVF cycle, tracking, by the processor, a change in the at least one uterine parameter, and predicting, by the processor, success of embryo implantation based on the change in the at least one uterine parameter.

In accordance with another aspect of the disclosure, the at least one uterine parameter includes an endometrium thickness, an endometrium volume, an endometrium pattern, a junctional zone thickness, a junctional zone volume, a uterine shape, and a uterine blood flow.

In accordance with another aspect of the disclosure, predicting the success of embryo implantation includes determining at least one of an implantation location in the uterus of the subject wherein an embryo is to be implanted, a time when the embryo is to be implanted, and uterine receptivity.

In accordance with another aspect of the disclosure, the method further comprises determining at least one of adenomyosis, endometriosis, structural anomalies in the uterus of the subject based on the change in the at least one uterine parameter.

In accordance with another aspect of the disclosure, the method further comprises determining a hormonal dosage for stimulation of the uterus of the subject within the IVF cycle, based on the change in the at least one of uterine parameter.

In accordance with another aspect of the disclosure, the method further comprises determining a guiding path of a needle for embryo implantation.

In accordance with another aspect of the disclosure, the method further comprises obtaining at least one uterine parameter of each of a plurality of subjects on predefined days of an IVF cycle of each of the plurality of subjects, comparing the at least one uterine parameter of the subject with the at least one uterine parameter of each of the plurality of subjects, and predicting success of embryo implementation based on a comparison result.

In accordance with another aspect of the disclosure, the one or more ultrasound images are obtained by obtaining, by the processor, a mid-coronal plane view of the uterus of the subject from a trans-vaginal ultrasound of the uterus of the subject and sequentially segmenting, by the processor, a plurality of uterine layers from the mid-coronal plane, wherein a last segmented uterine layer is a junctional zone of the uterus.

In accordance with another aspect of the disclosure, an apparatus for assessing uterine parameters comprises a memory storing at least one instruction, and a processor configured to execute the at least one instruction stored in the memory to determine at least one uterine parameter based on one or more ultrasound images of the uterus of a subject, the one or more ultrasound images being obtained at predefined days within an IVF cycle of the subject, track, a change in the at least one uterine parameter, predict success of embryo implantation based on the change in the at least one uterine parameter.

In accordance with another aspect of the disclosure, the at least one uterine parameter includes an endometrium thickness, an endometrium volume, an endometrium pattern, a junctional zone thickness, a junctional zone volume, and a uterine blood flow.

In accordance with another aspect of the disclosure, the processor is further configured to execute the at least one instruction to determine at least one of an implantation location in the uterus of the subject wherein an embryo is to be implanted, a time when the embryo is to be implanted, and uterine receptivity.

In accordance with another aspect of the disclosure, the processor is further configured to execute the at least one instruction to determine at least one of adenomyosis, endometriosis, structural anomalies in the uterus of the subject, based on the change in the at least one uterine parameter.

In accordance with another aspect of the disclosure, the processor is further configured to execute the at least one instruction to determine a path of a needle for embryo implantation.

In accordance with another aspect of the disclosure, the processor is further configured to execute the at least one instruction to obtain at least one uterine parameter of each of a plurality of subjects on predefined days of an IVF cycle of each of the plurality of subjects, compare the at least one uterine parameter of the subject with the at least one uterine parameter of each of the plurality of subjects, and predict success of embryo implementation based on a comparison result.

In accordance with another aspect of the disclosure, the one or more ultrasound images are obtained by obtaining a mid-coronal plane view of the uterus of the subject from a trans-vaginal ultrasound of the uterus of the subject, and sequentially segmenting a plurality of uterine layers from the mid-coronal plane, wherein a last segmented uterine layer is a junctional zone of the uterus.

The embodiments include generating a nomograph based on a comparison of a change in the at least one uterine parameter of the subject with a change in the at least one uterine parameter of each of the plurality of subjects.

These and other aspects of the embodiments herein will be better appreciated and understood when considered in conjunction with the following description and the accompanying drawings. It should be understood, however, that the following descriptions, while indicating embodiments and numerous specific details thereof, are given by way of illustration and not of limitation. Many changes and modifications may be made within the scope of the embodiments herein without departing from the spirit thereof, and the embodiments herein include all such modifications.

### MODE FOR THE INVENTION

The embodiments herein and various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The embodiments herein disclose methods of and systems for providing aid to clinicians for assessing uterine parameters. The embodiments include analyzing the rate of change of different uterine parameters during the In-Vitro Fertilization (IVF) cycle. The embodiments include obtaining a multi-layered scanned image (longitudinal scan) of a uterus of a subject, on predefined days within an IVF cycle of the subject for visualizing different layers of the uterus of the subject. The multi-layered scanned image may be used for determining the uterine parameters of the subject such as endometrium thickness, endometrium volume, junctional zone thickness, junctional zone volume, uterine blood flow, and other relevant uterine parameters; at the predefined days within the IVF cycle of the subject. Any changes in the uterine parameters, detected from the multi-layered scanned images obtained on the predefined days of the IVF cycle of the subject may be tracked. The embodiments include monitoring the rate of change of uterine parameters in the longitudinal scans. The embodiments include determining the implantation location and day for embryo transfer, and grading the uterus for embryo transfer. The embodiments include displaying an enhanced visualization of the implantation location. The uterine parameters of the subject on the predefined days of the IVF cycle may be compared with that of other subjects. The embodiments include generating nomographs based on the rate of change of the uterine parameters. The success of embryo implantation may be predicted based on the changes in the tracked uterine parameters during the IVF cycle of the subject. The method includes determining the hormonal dosage based on the tracked uterine parameters. The method includes classifying detected uterine shapes for determining structural anomalies. The method includes diagnosing uterine pathologies such as adenomyosis and endometriosis based on the quantified uterine parameters. The method includes segmenting the uterus for computing a uterus volume.

Referring now to the drawings showing preferred embodiments, reference characters denote corresponding features consistently throughout the figures.

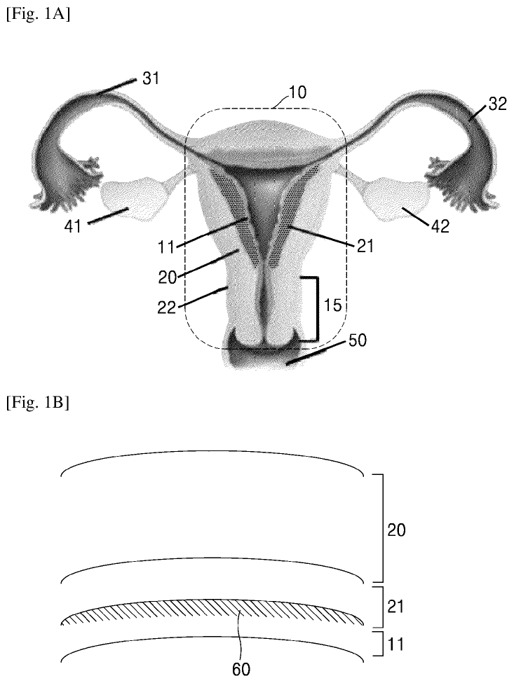

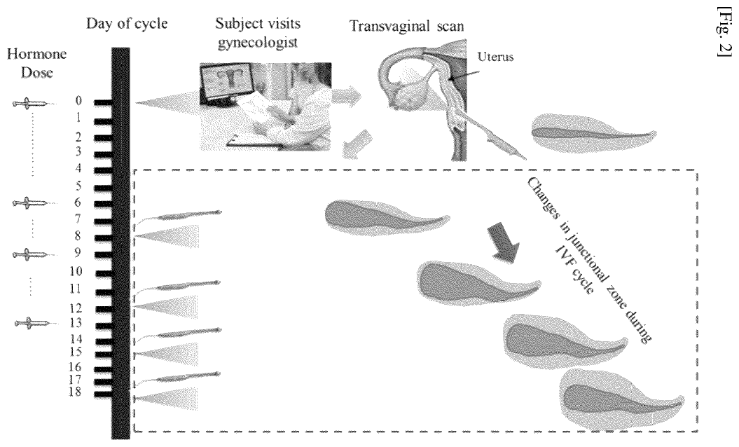

FIG. 1A depicts the anatomy of the female reproductive system. As depicted in FIG. 1A, the female reproductive system includes two ovaries (41, 42), two fallopian tubes (31, 32), uterus (10), serosa (22), cervix (15), and vagina (50). FIG. 1B depicts different layers of the uterus (10). The uterus (10) includes multiple layers, viz., endometrium (11), myometrium (20), and junctional zone (21). The innermost layer, which lines the uterine cavity, is the endometrium (11). The endometrium (11) is shed during the menstrual cycle. The myometrium (20) mostly includes muscle. The myometrium (20) may be further divided into an inner layer, which is the junctional zone (21), and an outer layer. The outermost layer of the uterus is the serosa (22), which is a very thin covering. In normal women, a line or region (60) dividing the endometrium (11) and the junctional zone (21) is generally clear and distinct.

The junctional zone (21) together with its overlying endometrium (11) is involved in placentation (pregnancy development). There is considerable variation in a thickness, volume, and appearance of the junctional zone (21), not only between individuals but also based on a hormonal status. The junctional zone (21) represents the vasculature of the myometrium (20), thus constituting a region of increased perfusion.

Further, a rate of change of junctional zone parameters such as a thickness, volume, appearance, and so on, may be determined by an ultrasound imaging system. The ultrasound imaging system transmits an ultrasound signal generated by a transducer of a probe to the uterus and receives information regarding an echo signal reflected from the uterus, thereby obtaining an image of a part inside the uterus. In particular, the ultrasound imaging system may used for medical purposes, such as internal observation of the uterus, diagnosis of damage in inside parts of the uterus, and so on.

FIG. 2 depicts a timeline of an IVF process, according to embodiments. When a subject approaches trained personnel, such as a gynecologist, for assisted reproduction or IVF, the trained personnel will scan the uterus of the subject by performing a 3D trans-vaginal ultrasound scan. The 3D trans-vaginal ultrasound may be performed on predefined days of the IVF cycle of the subject. The trained personnel may inject hormones for stimulation of the uterus during the predefined days within the IVF cycle.

The embodiments herein provide a method of automatically quantifying uterine parameters. A multi-layered scanned image of the uterus of the subject may be obtained from the 3D trans-vaginal ultrasound for visualizing different layers of the uterus. Similar multi-layered scanned images of the uterus may be obtained on predefined days within the IVF cycle of the subject. The uterine parameters such as an endometrium thickness, an endometrial volume, an endometrial pattern, a junctional zone thickness, a junctional zone volume, a uterine blood flow, and so on, may be quantified during each of the predefined days of the IVF cycle from the multi-layered scanned images (longitudinal scan) of the uterus. The uterine parameters may be observed, analyzed, and tracked throughout the IVF cycle to predict chances of successful embryo implantation. The prediction may be performed automatically. The uterine parameters may be tracked throughout the IVF cycle.

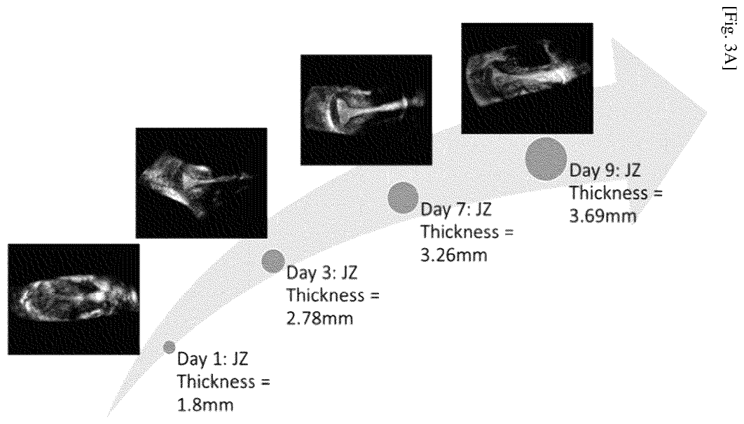

FIGS. 3A and 3B depict changes in the junctional zone (21) on predefined days of the IVF cycle, according to embodiments. The changes in a thickness of the junctional zone (21) on predefined days of the IVF cycle are depicted in FIG. 3A. The changes in a junctional zone volume on predefined days of the IVF cycle are depicted in FIG. 3B.

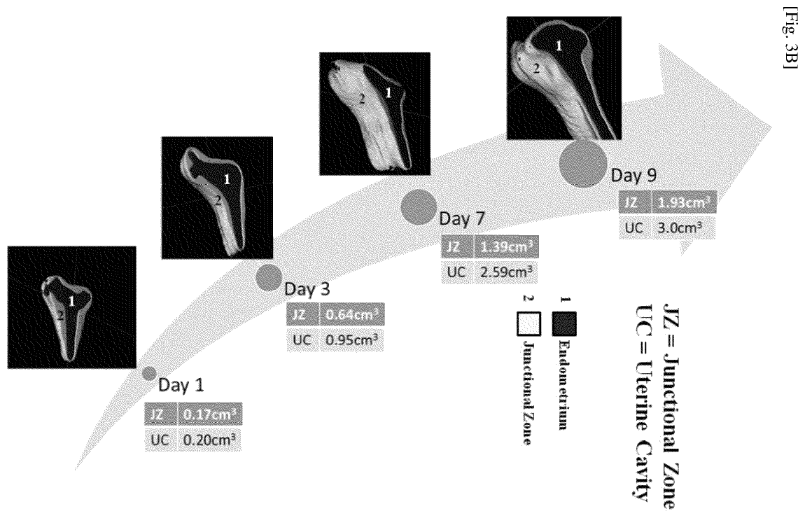

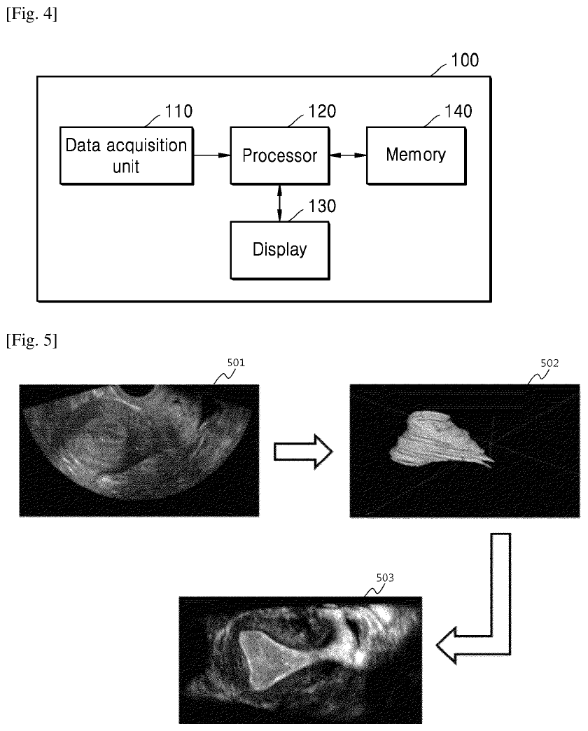

FIG. 4 is a block diagram of a configuration of an apparatus 100 for assessing uterine parameters, according to embodiments. Referring to FIG. 4, the apparatus 100 according to the present embodiment may include a data acquisition unit 110, a processor 120, a display 130, and a memory 140.

According to an embodiment, the data acquisition unit 110 may acquire medical image data with respect to an object. In an embodiment, the medical image data may include, but is not limited to ultrasound data In an embodiment, the data acquisition unit 110 may transmit ultrasound signals to the object and receive echo signals reflected by the object. The data acquisition unit 110 may generate ultrasound data with respect to the object by processing the received echo signals. In an embodiment, the data acquisition unit 110 may obtain ultrasound data from a 3D trans-vaginal ultrasound scan of the uterus of a subject.

In another embodiment, the data acquisition unit 110 may receive ultrasound data generated by an external ultrasound diagnosis apparatus, without directly generating medical image data by receiving an ultrasound signal.

According to an embodiment, the ultrasound data may be 2D data or 3D volume data. The 2D data may be data representing a cross-section of an object. Volume data indicates data obtained by stacking pieces of data representing cross-sections of the object and reconstructing the stacked pieces of data into a 3D format.

The processor 120 may control the apparatus 100. The processor 120 may execute at least one instruction stored in the memory 140.

The memory 140 may store various piece of data, programs, or applications for driving and controlling the apparatus 100. A program stored in the memory 140 may include at least one instruction. A program, at least one instruction, or an application stored in the memory 140 may be executed by the processor 120.

According to an embodiment, the processor 120 may generate a plurality of ultrasound images based on ultrasound data. In an embodiment, the processor 120 may generate at least one view across a plurality of planes of the uterus, viz., an axial plane, a sagittal plane, and a coronal plane. The mid-coronal plane view of the uterus may be helpful for observing and quantifying the uterine parameters for predicting the success of embryo implantation.

The processor 120 may preprocess the ultrasound image of the uterus and may perform endometrium segmentation on the ultrasound image of the uterus to obtain an endometrium mask. A surface fit may be, thereafter, performed on the endometrium mask in order to render the mid-coronal plane view of the uterus. The mid-coronal plane view is a 2D image which may be used to sequentially segment a plurality of uterine layers. In an embodiment, the uterine cavity may be segmented from the 2D mid-coronal plane view, followed by segmenting the junctional zone from the uterine cavity. A multi-layer scanned image of the uterus may be obtained after segmentation of the uterine cavity and the junctional zone (21).

The processor 120 may determine at least one of uterine parameters such as the endometrium thickness, the endometrium volume, the endometrial pattern, the junctional zone thickness, the junctional zone volume, the uterine blood flow, and other relevant uterine parameters from the multi-layer scanned image of the uterus of the subject. Different multi-layer scanned images may be obtained by performing 3D trans-vaginal ultrasound scans on predefined days within the IVF cycle of the subject.

The processor 120 may track changes in at least one of the uterine parameters, such as the endometrium thickness, the endometrium volume, endometrial pattern, the junctional zone thickness, the junctional zone volume, the uterine blood flow, and other uterine parameters on the predefined days within the IVF cycle of the subject. The chances of successful implantation of the embryo may be ascertained based on the changes in the tracked uterine parameters.

The processor 120 may determine the day and implantation location for embryo transfer based on the changes in the tracked uterine parameters.

In an embodiment, the chances of successful embryo implantation may be predicted by determining at least one of an implantation location in the uterus to implant the embryo, the day within the IVF cycle when the embryo may be implanted, uterine receptivity, and so on, which may be determined based on at least one of the uterine parameters tracked during the IVF cycle of the subject.

In an embodiment, the processor 120 may obtain uterine parameters such as an endometrium thickness, an endometrium volume, an endometrium pattern, a junctional zone thickness, a junctional zone volume, a uterine blood flow, and other uterine parameters of other subjects from a database. The uterine parameters of the other subjects may be compared with that of the determined uterine parameters of the subject over the IVF cycle period to generate a report and nomographs. The processor 120 may predict the chances of successful embryo implantation based on a comparison result.

The display 130 may generate a driving signal by converting an image signal, a data signal, an on-screen display (OSD) signal, and a control signal that are processed by the processor 120. The display 130 may be a plasma display panel (PDP), a liquid-crystal display (LCD), an organic light-emitting device (OLED), a flexible display, or a 3-dimensional (3D) display. The display 130 may be configured as a touch screen, and thus may serve as an input device as well as an output device.

According to an embodiment, the display 130 may display the multi-layered image of the uterus. The display 130 may display the quantified uterine parameters, nomographs (which are generated using the quantified uterine parameters), reports, and so on. The display 130 may display the junctional zone (21) of the uterus on the ultrasound image of the uterus. The display 130 may display at least one of an implantation location in the uterus where an embryo is to be implanted, a time when the embryo is to be implanted, and uterine receptivity. The display 130 may display a path of a needle for embryo implantation.

FIG. 4 shows exemplary elements of the apparatus 100, but it is to be understood that other embodiments are not limited thereto. In other embodiments, the apparatus 100 may include less or more elements. Further, the labels or names of the elements are used only for illustrative purpose and do not limit the scope of the embodiments herein. One or more elements may be combined together to perform same or substantially similar function in the apparatus 100.

FIG. 5 depicts rendering of a mid-coronal plane view of the uterus from a 3D trans-vaginal ultrasound image, according to embodiments. 2D imaging provides information through axial and sagittal planes and may be limited due to inaccessibility of a coronal plane. One of the main advantages of 3D imaging of the uterus is the ability to reconstruct the mid-coronal plane. An enhanced visualization of the uterine parameters, especially the junctional zone (21), which is an important and critical parameter for predicting success of embryo implantation, may be obtained using the mid-coronal plane view of the uterus. In order to accurately assess the boundary and shape of the endometrium and junctional zone, clinicians may extract the true mid-coronal plane view of the uterus from the 3D trans-vaginal ultrasound image. The mid-coronal plane allows visualization and enables delineation of layers in the uterus (the endometrium (11), the myometrium (20), and the junctional zone (21)). As depicted in FIG. 5, endometrium segmentation may be performed on the 3D trans-vaginal ultrasound image (501). An endometrium mask (502) may be obtained by performing endometrium segmentation, which may be, thereafter, surface fitted to render a mid-coronal plane view (503).

In an embodiment, the endometrium segmentation may be performed using a convolution neural network, the surface fitting may be performed using least square energy minimization, and the mid-coronal plane rendering may be performed using ray compositing, but the embodiments are not limited thereto.

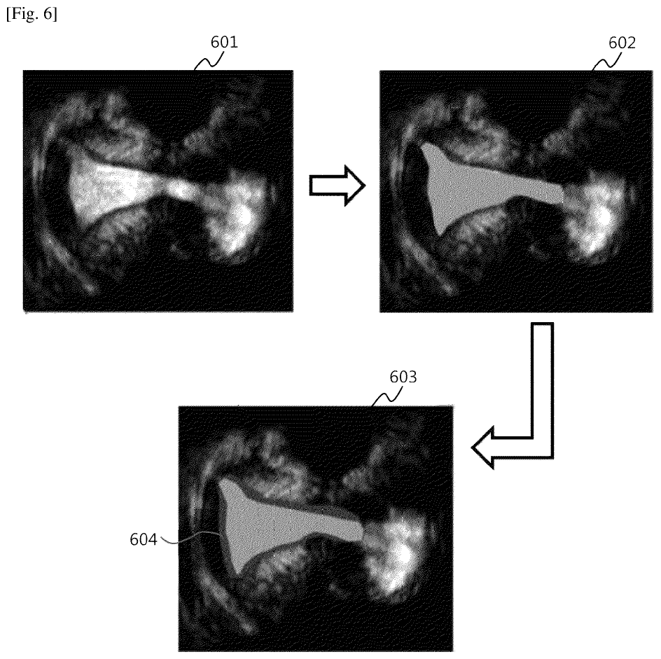

FIG. 6 depicts segmentation of junctional zone (21) of the uterus from the mid-coronal plane view (503), according to embodiments. Responsiveness, thickness, and volume of the junctional zone during the IVF period may be used to predict the outcome of embryo implantation. It may not be possible to predict the likelihood of pregnancy using IVF by measuring the endometrial thickness, endometrial volume, and endometrial pattern, at the time of implanting the embryo during the IVF cycle. However, changes in other uterine layers such as the junctional zone (21) and responsiveness in conception cycles may be more relevant than in non-conception cycles. The responsiveness of the junctional zone (21) as a marker for predicting the success of embryo implantation may be more precise.

As depicted in FIG. 6, the junctional zone (21) may be visible as a halo (604) around the endometrium (11). The mid-coronal plane view is a 2D image (601) of the uterus. Thereafter, segmentation is performed sequentially at multiple levels on the 2D image of the uterus. In an embodiment, the 2D image may be chosen as a seed to segment the uterine cavity. An image (602) with the uterine cavity segmented is obtained. Thereafter, the uterine cavity may be chosen as a seed and the junctional zone (21) may be segmented to obtain a multi-layer segmented (scanned) image. Once the multi-layer scanned image is obtained (603), the multi-layer scanned may be used for quantification of uterine parameters.

The multi-layer segmentation of the uterine layers may be performed by one of an active contour, a region based segmentation, a morphology based segmentation, and so on.

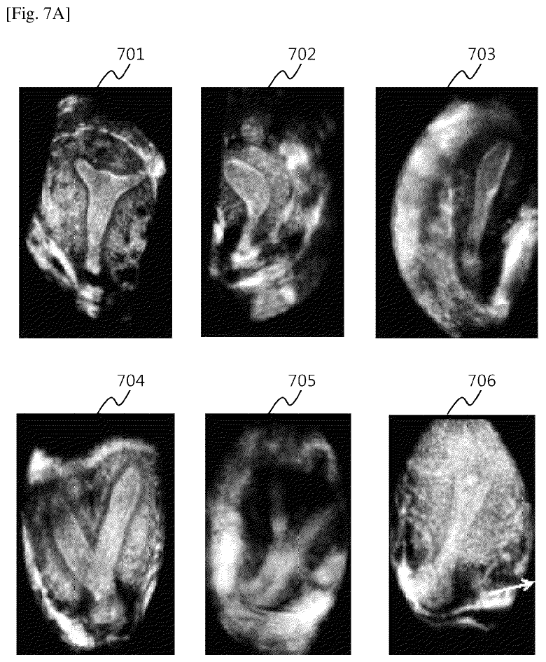

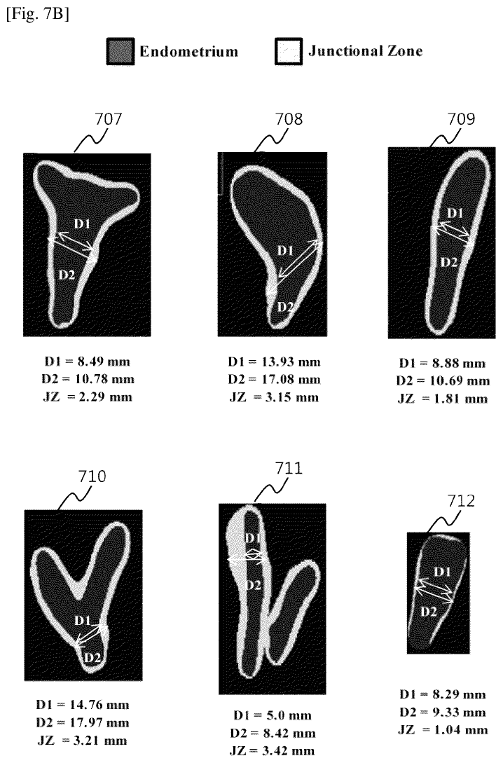

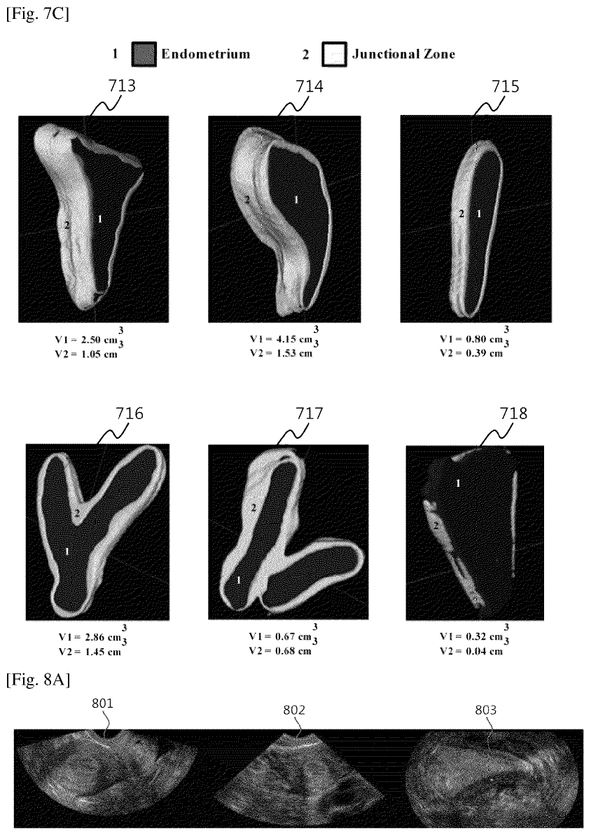

FIGS. 7A-7C depict results of segmentation of the uterine cavity and the junctional zone (21), according to embodiments. Numerals 701-706 depict different mid-coronal plane views obtained, numerals 707-712 depict multi-layer segmentation obtained after segmenting the uterine cavity and the junctional zone (21). Numerals 713-718 depict 3D volume rendering of the uterine cavity and the junctional zone (21), obtained after segmenting the uterine cavity and the junctional zone (21) in multiple coronal planes around the mid-coronal plane.

The classified uterine shapes are as depicted in FIGS. 7A-7C. Classification of different uterine shapes such as normal uterus (701, 707, 713), diverted uterus (702, 708, 714), unicornuate uterus (703, 709, 715), bicornuate uterus (704, 710, 716), and didelphys uterus (705, 711, 717), may be used for determining structural anomalies present in the uterus.

The uterine pathologies such as adenomyosis and endometriosis may be diagnosed.

In general adenomyosis (706), the junctional zone (21) is breached. The junctional zone (21) may be represented as a very thin lining around the uterine cavity (712). Additionally, the junctional zone volume may be very low due to the breach (718).

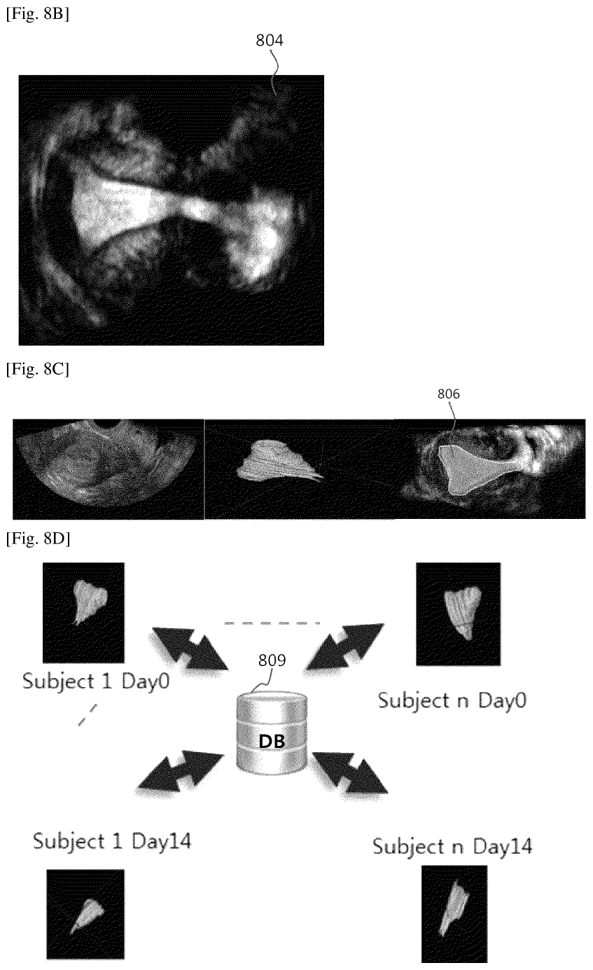

FIGS. 8A-8E depict tracking of the junctional zone during the IVF cycle, according to embodiments. As depicted in FIG. 8A, three plane views (801, 802, 803) of the uterus, viz., axial plane, sagittal plane, and a coronal plane, are obtained by carrying out a 3D trans-vaginal ultrasound scan on the uterus of a subject.

From the 3D trans-vaginal ultrasound scan, as depicted in FIG. 8B, a mid-coronal plane (804) of the uterus may be obtained. Enhanced visualization of different uterine layers may be obtained. Multiple layers of the uterus may be segmented sequentially from the mid-coronal plane view of the uterus.

As depicted in FIG. 8C, the final layer (806) which may be segmented in the segmentation sequence starting from the mid-coronal plane view may be the junctional zone (21) of the uterus. The multi-layer scanned image of the uterus, with all its layers (uterine cavity and junctional zone) segmented, may be utilized for determining the uterine parameters. The uterine parameters may be determined by quantifying them using the multi-layer scanned image. The 3D trans-vaginal ultrasound scan may be performed on predefined days within the IVF cycle. The uterine parameters may be thus quantified on the predefined days and changes in the uterine parameters may be tracked. The uterine parameters may be an endometrium thickness, an endometrium volume, an endometrium pattern, a junctional zone thickness, a junctional zone volume, a uterine blood flow, and other relevant uterine parameters.

As depicted in FIG. 8D, the uterine parameters obtained from other subjects at similar stages (days) within the IVF cycle may be compared with the determined uterine parameters of the subject. The uterine parameters of the other subjects may be obtained from a database (809). The uterine parameters of the subject may also be stored in the database (809) for future use. Embodiments herein provide automated tracking and quantification of the changes in at least one of uterine parameters during the IVF cycle.

As depicted in FIG. 8E, the success of embryo implantation may be predicted based on at least one of the quantified uterine parameters of the subject tracked throughout the IVF cycle and the uterine parameters of the other subjects, which are retrieved from the database (809) and compared with the tracked uterine parameters of the subject. The prediction of the success of embryo implantation includes determining at least one of the implantation location in the uterus in which the embryo is to be implanted, a day within the IVF cycle when the embryo is to be implanted, uterine receptivity, and so on, which are determined based on the uterine parameters of the subject quantified and tracked throughout the IVF cycle.

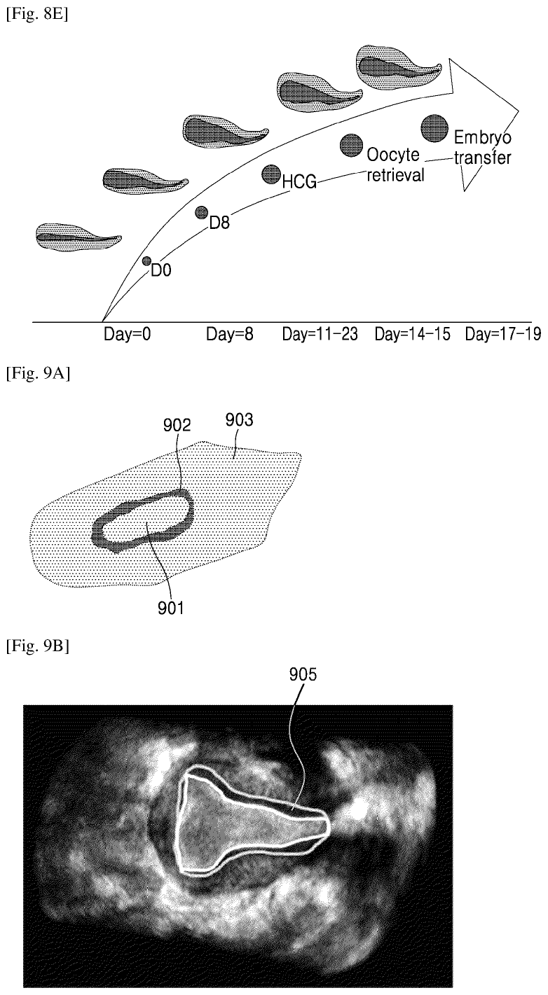

FIGS. 9A and 9B depict enhanced visualization of perfusion in the junctional zone (21), according to embodiments. The sagittal view and the coronal view of the junctional zone (21) of the uterus are depicted in FIG. 9A and FIG. 9B, respectively. Referring FIG. 9A, a reference numeral 901 denotes the endometrium, a reference numeral 902 denotes the junctional zone, and a reference numeral 903 denotes the outer myometrium. The views allow discerning the growth, the thickness, and the volume of the junctional zone, and the blood flow (905) inside the junctional zone. The measurement or quantification of the junctional zone may be graded to estimate whether the embryo implantation will be successful. Utilizing the quantified junctional zone parameters for predicting the success of embryo implantation may increase the accuracy of prediction.

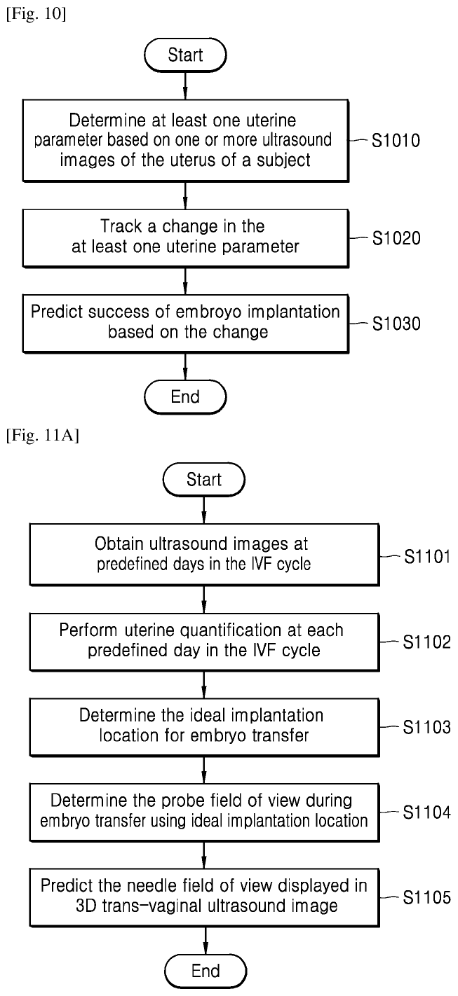

FIG. 10 shows a flowchart of a method of assessing uterine parameters via the apparatus 100, according to an embodiment.

Referring to FIG. 10, in step S1010, the apparatus 100 may determine at least one uterine parameter based on one or more ultrasound images of the uterus of the subject. In an embodiment, the apparatus 100 may determine at least one uterine parameter such as the endometrium thickness, the endometrium volume, the endometrial pattern, the junctional zone thickness, the junctional zone volume, the uterine blood flow, and other relevant uterine parameters from the multi-layer scanned image of the uterus of the subject. Different multi-layer scanned images may be obtained by performing 3D trans-vaginal ultrasound scans on predefined days within the IVF cycle of the subject. In step S1020, the apparatus 100 may track a change in the at least one uterine parameter. In an embodiment, the apparatus 100 may track changes in at least one of the uterine parameters, such as the endometrium thickness, the endometrium volume, the endometrial pattern, the junctional zone thickness, the junctional zone volume, the uterine blood flow, and other uterine parameters on the predefined days within the IVF cycle of the subject. The chances of successful implantation of the embryo may be ascertained based on the changes in the tracked uterine parameters.

In step S1030, the apparatus 100 may predict success of embryo implementation based on the change. In an embodiment, the apparatus 100 may determine the day and implantation location for embryo transfer based on the changes in the tracked uterine parameters. The chances of successful embryo implantation may be predicted by determining at least one of an implantation location in the uterus where the embryo may be implanted, the day within the IVF cycle when the embryo may be implanted, uterine receptivity, and so on, which may be determined based on at least one of the uterine parameters tracked during the IVF cycle of the subject.

The apparatus 100 may obtain the uterine parameters such as the endometrium thickness, the endometrium volume, the endometrium pattern, the junctional zone thickness, the junctional zone volume, the uterine blood flow, and other uterine parameters of other subjects from a database. The uterine parameters of the other subjects may be compared with that of the determined uterine parameters of the subject over the IVF cycle period to generate a report and nomographs. The apparatus 100 may then predict the chances of successful embryo implantation based on a comparison result.

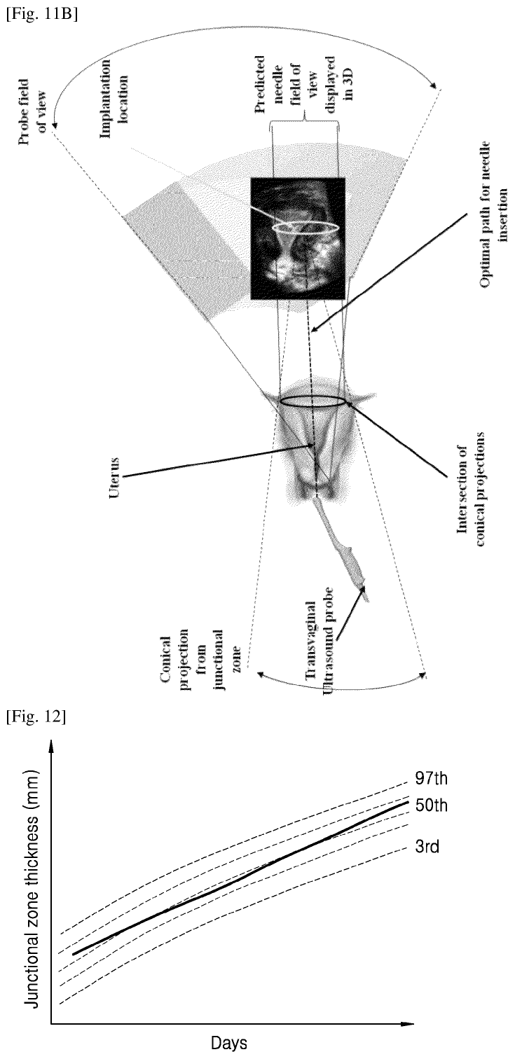

FIG. 11A depicts an example flowchart of a method of tracking a needle path for embryo implantation, according to embodiments. In step S1101, the method includes obtaining a 3D trans-vaginal ultrasound image of a uterus of a subject on predefined days of the IVF cycle of the subject. In step S1102, the method includes performing quantification of uterine parameters on predefined days in the IVF cycle of the subject. In step S1103, the method includes predicting an appropriate location in the uterus for implantation of an embryo. In step S1104, the method includes determining a field of view of a probe based on the predicted location for implantation of the embryo. In step S1105, the method includes predicting a field of view of a needle to be displayed in the 3D trans-vaginal ultrasound image.

The various steps of the method may be performed in the order presented, in a different order, or simultaneously. Further, in some embodiments, some steps listed in FIG. 11A may be omitted.

FIG. 11B is an example scenario depicting needle path tracking for embryo implantation, according to an embodiment. In an embodiment, the appropriate implantation location for embryo transfer is determined by analyzing the multi-layer scanned image of the uterus obtained from the 3D trans-vaginal ultrasound image.

FIG. 12 illustrates a graph depicting a nomograph obtained from tracked uterine parameters, according to embodiments. The nomograph may be a plot of ‘a thickness of the junctional zone’, ‘a volume of the junctional zone’, ‘an endometrium thickness’, ‘an endometrium volume’ or other relevant uterine parameters with respect to ‘day in the IVF cycle’. The uterine parameters are quantified on predefined days within the IVF cycle a plurality of subjects. Based on the quantified values on the predefined days for the plurality of subjects, nomographs may be generated and displayed to predict the success of embryo transfer.

The embodiments disclosed herein may be implemented through at least one software program running on at least one hardware device and performing network management functions to control the network elements. The network elements shown in FIG. 4 include blocks which may be at least one of a hardware device or a combination of hardware device and software module.

The embodiments disclosed herein describe methods and systems for providing clinical aid to gynecologists for predicting success of embryo implantation by assessing uterine parameters. Therefore, it is understood that the scope of the protection is extended to such a program and in addition to a computer readable means having a code therein for implementation of one or more steps of the method, when the program runs on a server or mobile device or any suitable programmable device. The method is implemented in a preferred embodiment through or together with a software program written in e.g. Very High Speed Integrated Circuit Hardware Description Language (VHDL) or another programming language, or implemented by one or more VHDL or several software modules being executed on at least one hardware device. The hardware device may be any kind of portable device that may be programmed. The device may also include means such as hardware means like e.g. an ASIC, or a combination of hardware and software means, e.g. an ASIC and an FPGA, or at least one microprocessor and at least one memory with software modules located therein. The method embodiments described herein may be implemented partly in hardware and partly in software. Alternatively, the invention may be implemented on different hardware devices, e.g., using a plurality of CPUs.

Also, a method of or an apparatus for assessing uterine parameters according to the disclosed embodiments may be provided in the form of a computer program product. The computer program product may be traded as a commodity between a seller and a purchaser.

The computer program product may include a software program and a computer-readable storage medium having the software program stored thereon. In an embodiment, the computer program product may include a product in the form of a software program such as a downloadable app that may be electronically distributed through a manufacturer of an apparatus or an electronic market. For electronic distribution, at least a portion of the software program may be stored on a storage medium or may be created temporarily. In this case, the storage medium may be a server of a manufacturer, a server of an electronic market, or a storage medium of a relay server for temporarily storing a software program.

The computer program product may include, in a system including a server and a client device (for example, an apparatus according to the disclosed embodiments), a storage medium of the server or a storage medium of the client device. Alternatively, when a third device (e.g., a smartphone) is in communication with the server or a client device, the computer program product may include a storage medium of the third device. Alternatively, the computer program product may include the software program itself transmitted from the server to the client device or the third device, or transmitted from the third device to the client device.

In this case, one of the server, the client device, and the third device may execute the computer program product to perform the methods according to the disclosed embodiments. Alternatively, at least two of the server, the client device, and the third device may execute the computer program product to distribute and perform the methods according to the disclosed embodiments.

In an embodiment, a server (e.g., a cloud server or an artificial intelligence server) may execute a computer program product stored on a server to control a client device communicating with the server to perform the methods according to the disclosed embodiments.

The foregoing description of the specific embodiments reveals the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A method of assessing uterine parameters via an apparatus including a processor, the method comprising:
determining, by the processor, at least one uterine parameter based on one or more ultrasound images of the uterus of a subject, the one or more ultrasound images being obtained at predefined days within an IVF cycle of the subject;
tracking, by the processor, a change in the at least one uterine parameter; and
predicting, by the processor, success of embryo implantation based on the change in the at least one uterine parameter.

2. The method of claim 1, wherein the at least one uterine parameter includes an endometrium thickness, an endometrium volume, an endometrium pattern, a junctional zone thickness, a junctional zone volume, a uterine shape, and a uterine blood flow.

3. The method of claim 1, wherein predicting the success of embryo implantation includes determining at least one of an implantation location in the uterus of the subject wherein an embryo is to be implanted, a time when the embryo is to be implanted, and uterine receptivity.

4. The method of claim 1, wherein the method further comprises:
determining at least one of adenomyosis, endometriosis, and structural anomalies in the uterus of the subject based on the change in the at least one uterine parameter.

5. The method of claim 1, wherein the method further comprises:
determining a hormonal dosage for stimulation of the uterus of the subject within the IVF cycle, based on the change in the at least one of uterine parameter.

6. The method of claim 1, wherein the method further comprises:
determining a path of a needle for the embryo implantation.

7. The method of claim 1, wherein the method further comprises:
obtaining at least one uterine parameter of each of a plurality of subjects on predefined days of an IVF cycle of each of the plurality of subjects;
comparing the at least one uterine parameter of the subject with the at least one uterine parameter of each of the plurality of subjects, and predicting success of embryo implementation based on the comparison result.

8. The method of claim 1, wherein the one or more ultrasound images are obtained by:
obtaining, by the processor, a mid-coronal plane view of the uterus of the subject from a trans-vaginal ultrasound of the uterus of the subject; and
sequentially segmenting, by the processor, a plurality of uterine layers from the mid-coronal plane, wherein the last segmented uterine layer is a junctional zone of the uterus of the subject.

9. An apparatus for assessing uterine parameters, the apparatus comprising:
a memory storing at least one instruction; and
a processor configured to execute the at least one instruction stored in the memory to:
determine, at least one uterine parameter based on one or more ultrasound images of the uterus of a subject, the one or more ultrasound images being obtained on predefined days within an IVF cycle of the subject;
track a change in the at least one uterine parameter;
predict success of embryo implantation based on the change in the at least one uterine parameter.

10. The apparatus of claim 9, wherein the at least one uterine parameter includes an endometrium thickness, an endometrium volume, an endometrium pattern, a junctional zone thickness, a junctional zone volume, and a uterine blood flow.

11. The apparatus of claim 9, wherein the processor is further configured to execute the at least one instruction to:
determine at least one of an implantation location in the uterus of the subject wherein an embryo is to be implanted, a time when the embryo is to be implanted, and uterine receptivity.

12. The apparatus of claim 9, wherein the processor is further configured to execute the at least one instruction to:
determine at least one of adenomyosis, endometriosis, and structural anomalies in the uterus of the subject, based on the change in the at least one uterine parameter.

13. The apparatus of claim 9, wherein the processor is further configured to execute the at least one instruction to determine a path of a needle for the embryo implantation.

14. The apparatus of claim 9, wherein the processor is further configured to execute the at least one instruction to:
obtain at least one uterine parameter of each of a plurality of subjects on predefined days of an IVF cycle of each of the plurality of subjects;
compare the at least one uterine parameter of the subject with the at least one uterine parameter of each of the plurality of subjects, and predict success of embryo implementation based on a comparison result.

15. The apparatus of claim 9, wherein the one or more ultrasound images are obtained by:
obtaining a mid-coronal plane view of the uterus from a trans-vaginal ultrasound of the uterus of the subject; and
sequentially segmenting a plurality of uterine layers from the mid-coronal plane, wherein a last segmented uterine layer is a junctional zone of the uterus of the subject.
